(19) United States
(12) Patent Application Publication
(10) Pub. No.: US 2007/0104740 A1
(43) Pub. Date: May 10, 2007

(54) Self-microemulsifying drug delivery systems of a hiv protease inhibitor

(76) Inventor: Jody Firmin Marceline Voorspoels, Reningelst (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/596,738

(22) PCT Filed: Dec. 23, 2004
(86) PCT No.: PCT/EP04/53700

§ 371 Date: Jun. 22, 2006

Related U.S. Application Data
(60) Provisional application No. 60/565,451, filed on Apr. 26, 2004.

(30) Foreign Application Priority Data
Dec. 23, 2003 (EP) 03104959.6

(51) Int. Cl. A61K 31/343; A61K 9/00

(52) U.S. Cl. 424/400; 514/469

(57) ABSTRACT

The present invention relates to pharmaceutical formulations of (3R,3aS,6aR)-hexahydrofuro[2,3-b]furan-3-yl(1S,2R)-3-[[(4-aminophenyl)sulfonyl](isobutyl) amino]-1-benzyl-2-hydroxypropylcarbamate, salts, esters, polymorphic and pseudopolymorphic forms thereof, which are self-microemulsifying drug delivery systems and comprise as carrier a lipophilic phase, one or more surfactants, a hydrophilic solvent and a nucleation inhibitor.

### TECHNICAL FIELD

The present invention relates to the field of drug delivery systems, in particular to the field of self-microemulsifying drug delivery systems. These systems have the property of forming spontaneously a microemulsion upon contact with an aqueous environment. The present invention further concerns (3R,3aS,6aR)-hexahydrofuro[2,3-b]furan-3-yl (1S,2R)-3-[[(4-aminophenyl)sulfonyl](isobutyl)amino]-1-benzyl-2-hydroxypropyl-carbamate, an HIV protease inhibitor, formulated in self-microemulsifying drug delivery systems.

### BACKGROUND INFORMATION

(3R,3aS,6aR)-hexahydrofuro[2,3-b]furan-3-yl(1S,2R)-3-[[(4-aminophenyl)sulfonyl](isobutyl) amino]-1-benzyl-2-hydroxypropylcarbamate has HIV protease inhibitory activity and is particularly well suited for inhibiting HIV-1 replication.

(3R,3aS,6aR)-hexahydrofuro[2,3-b]furan-3-yl(1S,2R)-3-[[(4-aminophenyl)sulfonyl](isobutyl) amino]-1-benzyl-2-hydroxypropylcarbamate, referred herein further as compound (I), and processes for its preparation are disclosed in EP 715618, WO 99/67417, U.S. Pat. No. 6,248,775, and in Bioorganic and Chemistry Letters, Vol. 8, pp. 687-690, 1998, “Potent HIV protease inhibitors incorporating high-affinity P₂-ligands and (R)-(hydroxyethylamino)sulfonamide isostere”. Pseudopolymorphic forms of compound (I) have also been described in WO 03/106461, all of which are incorporated herein by reference.

Like many of recently discovered chemical entities, one of the properties of compound (I) is its poor water solubility. For instance, the ethanolate form of compound (I) exhibits an aqueous solubility of approximately 0.18 mg/ml at a pH=2, which is considered to be very slightly soluble according to Ph. Eur. (European Pharmacopeia) and USP (United States Pharmacopeia). Aqueous solubility is often found to be among the most important factors affecting bioavailability, as an insufficient aqueous solubility results in erratic or incomplete absorption, thus producing a less than desirable therapeutic response.

Combination regimens are known to show potent antiretroviral activity and are referred to as HAART (highly active antiviral therapy) and are therefore extensively recommended. In this respect, WO03/049746 discloses a combination of a therapeutically effective amount of a hexahydrofuro[2,3-b]furanyl containing HIV protease inhibitor, and a therapeutically effective amount of a cytochrom P450 inhibitor. However, one of the few drawbacks of these regimens is the increase in pill burden experienced by the patients. The administration of highly loaded dosage forms is thus more desirable than the higher frequency of administration of less loaded formulations.

Lipid-based formulations have shown their utility to enhance the absorption of poorly absorbable drugs, especially emulsified formulations (Humberstone and Charman, 1997, Elsevier Science; Charman 2000, Jour. Pharm. Sci., vol. 89, no. 8), acting on physicochemical mechanisms, like increasing the solubilisation capacity of the gastrointestinal tract. Self-emulsifying drug delivery systems and self-microemulsifying drug delivery systems have been previously described in the literature as homogeneous mixtures of natural or synthetic oils, solid or liquid surfactants, or alternatively, one or more lipophilic solvents and co-solvents (Constantinides, Pharm. Res. 12 (1995) 1561-1572). The principal characteristic of these systems is their ability to form fine water-in-oil (w/o) or oil-in-water (o/w) emulsions or microemulsions upon mild agitation following dilution by lipophilic or aqueous phases, respectively. Self-emulsifying drug delivery systems and self-microemulsifying drug delivery systems are further considered suitable compositions for preparing high dosage pre-concentrates without increasing the overall weight of the drug delivery system.

Although several self-emulsifying drug delivery system formulations have been described in the literature, for instance self-microemulsifying drug delivery systems of 5,6-dihydro-4-hydroxy-2-pyrone sulfonamide inhibitors, there remains a challenge for the pharmaceutical formulator to predict which oil(s) and surfactant(s) to select for a particular application, taking as well into consideration their acceptability due to potential toxicity (E. C. Swenson and W. J. Curatolo, Adv. Drug Deliv. Rev. 8:39-93 (1992)). Furthermore, in the particular case of preparing increased dosages of compound (I), other parameters such as the avoidance of drug crystallization and precipitation need to be considered, while ensuring acceptable drug levels reaching the systemic circulation to effect the desired therapeutic response. There is a need therefore, for improved and viable oral formulations of compound (I), which exhibit a suitable oral bioavailability, can sustain an appropriate drug load and are acceptably stable.

Taken into account the previous limiting factors, the inventors have surprisingly found that compound (I) is able to form spontaneous microemulsions when compounded with certain self-microemulsifying drug delivery system excipients. These microemulsions have advantageously demonstrated increased rates of absorption of the drug, consequently enhancing its bioavailability.

Furthermore, it has also been found that by compounding a nucleation inhibitor and a hydrophilic solvent into the self-microemulsifying drug delivery systems of the present invention, the solubility of the drug in the pharmaceutical carrier is significantly increased, while minimizing the risk of drug precipitation. As such, said improvements allow an increase in the drug load as well as providing sufficient stability for the drug in these dosage forms.

While on the one hand, nucleation inhibitors increase the viscosity of preconcentrates, thus making less favourable the formation of emulsions, on the other hand, the addition of hydrophilic solvents to the preconcentrates confer a decrease in the bioavailablility of the drug. In this respect, U.S. Pat. No. 6,008,228 by Hoffmann La Roche discloses self-microemulsifying compositions that increase the bioavailability of a proteinase inhibitor, said compositions comprising a proteinase inhibitor, an ester of an alcohol with C₈₋₁₀ fatty acids, such as Capmul MCM, a hydrophilic surfactant system such as Cremophor or Labrasol, an hydrophilic solvent such as PEG 400 in amounts ranging from 0 to 28%, and a nucleation inhibitor such as PVP K30 in amounts ranging from 0 to 30%, preferably between 20 and 30% by weight.

Surprisingly in the present invention, by combining a hydrophilic solvent in a range of 1% (w/w) to 60% (w/w) and a nucleation inhibitor in a range of 0.1% (w/w) to 4% (w/w), the formulation thereof has proved advantageous when compared to the prior art by increasing the solubility and minimizing precipitation of the drug. In addition, said combination has challenged the prejudice of the state of the art which recommends the use of each of these two excipients separately.

Furthermore, the proposed formulations although containing an alcohol-based solvent, do not present the disadvantages exhibited by the encapsulated self-emulsifying drug delivery systems and self-microemulsifying drug delivery systems of the state of art wherein the alcohol migrates to the capsule cover thereby producing brittleness. Whereas the state of the art eliminates or diminishes the amounts of the alcohol-based hydrophilic solvent system, the present invention has included alcohol-based solvent without jeopardizing the stability of the capsules. As well, the capsules containing the self-microemulsifying drug delivery system of the present invention do not exhibit a tendency to soften and to stick to one another over time.

In addition, components of the present formulation possess satisfactory processing properties, while requiring basic mixing equipment. The present invention thus allows the economical production and processing of physiochemically stable and pharmaceutically acceptable oral dosage forms.

US20030044434 by Gao et al. concerns a self-emulsifying formulation for lipophilic compounds, which comprises a lipophilic, pharmaceutically active agent, a mixture of diglyceride and monoglyceride of-unsaturated fatty acid esters having sixteen to twenty-two carbon chain length, one or more pharmaceutically acceptable solvents, and one or more pharmaceutically acceptable surfactants.

EP 1170003 by Hovid Sdn Bhd relates to a formulation for fat-soluble drugs which self-emulsify in the presence of an aqueous medium with little agitation, comprising a mixture of drug with an appropriate oil and an appropriate surfactant system.

JP 2001151669 by Nippon Kayaku Co Ltd. discloses a self-emulsifiable preparation for oral administration. Components include 20-50 weight (wt.) % of fatty acid ester of glycerin and/or fatty acid ester of propylene glycol, 10-60 wt. % of a surfactant, 10-60 wt. % of a polar organic solvent and 0.1-30 wt. % of a medicinal ingredient.

WO01/091727 by Basf AG discloses a self-emulsifying formulation comprising one active substance; a lipid component; a bonding agent component; and if necessary, further auxiliary materials. The lipid component is selected from fatty acids, triglycerides, diglycerides and monoglycerides, and exhibits an HLB (hydrophilic-lipophilic balance) value of at most 12, preferably from 8 to 5. The bonding agent component is selected from polyvinylpyrrolidone, vinylpyrrolidone vinyl acetate copolymers, hydroxyalkylcellulose, hydroxyalkyl alkylcellulose, cellulosephthalate, polyalkylenglycol, and (meth)acrylate.

WO00/033862) by Pharmasolutions Inc discloses a pharmaceutical composition comprising a lipophilic drug in association with a propylene glycol ester of C6-C18 fatty acid having at least about 60% by weight of monoester based on the total weight of the propylene glycol ester; and a non-ionic surfactant, said non-ionic surfactant being present in an amount sufficient to form a microemulsion with the propylene glycol ester and drug when brought into contact with an aqueous medium.

U.S. Pat. No. 5,993,858 by Port Systems L.L.C. relates to a method and formulation which includes an emulsion including an oil or other lipid material, a surfactant, and a hydrophilic co-surfactant, and drugs formulated thereby.

WO95/08983 by GattefosséETS SA relates to a pharmaceutical composition forming a microemulsion comprising one active ingredient, a lipophilic phase, a surfactant, a co-surfactant, a hydrophilic phase.

WO02/361 10 by Boehringer Ingelheim Pharmaceuticals, Inc. relates to a microemulsion of pyranone protease inhibitor compounds that is substantially free of alcohol and propylene glycol comprising a pyranone protease inhibitor, one or more pharmaceutically acceptable surfactants, and a polyethylene glycol solvent, and a lipophilic component comprising medium chain mono- and di-glycerides, and optionally a basic amine.

WO99/06043 by Upjohn Co. discloses a self-emulsifying formulation which comprises pyranone compounds, a mixture of diglyceride and monoglyceride, one or more solvents and one or more surfactants. WO99/06044 also by Upjohn Co. discloses a self-emulsifying formulation which comprises as well pyranone compounds, a basic amine, one or more solvents and one or more surfactants.

WO98/22106 by Abbott Laboratories discloses an oral liquid self-emulsifying pharmaceutical composition for inhibitors of HIV protease. Such composition comprises a long-chain fatty acid composition, and a pharmaceutically acceptable alcohol, and optionally a surfactant (such as Cremophor EL, BASF Corp.).

WO96/39142 by Hoffmann La Roche teaches a pharmaceutical composition of protease inhibitors. The composition include a pharmaceutically acceptable carrier comprising monoglycerides of medium chain-saturated C6 to C12 fatty acids.

WO95/07696 also by Abbott Laboratories describes a pharmaceutical composition comprising a solution of an HIV protease inhibiting compound in a pharmaceutically acceptable organic solvent, the solvent comprising a pharmaceutically acceptable alcohol. The solution can optionally be encapsulated in a hard gelatin capsule or a soft elastic gelatin capsule. The composition can optionally comprise a pharmaceutically acceptable acid. The composition can optionally comprise an additive or a mixture of additives independently selected from glycerin, pharmaceutically acceptable surfactants and antioxidants.

### SUMMARY OF THE INVENTION

The present invention provides a pharmaceutical formulation comprising
(a) a therapeutically effective amount of (3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate, salts, esters, polymorphic and pseudopolymorphic forms thereof, and
(b) a carrier comprising
   esters of alcohols with C₆₋₁₂ fatty acids or oils;
   a hydrophilic surfactant system;
   a nucleation inhibitor; and
   a hydrophilic solvent.

The present invention provides as well dosage forms which may incorporate said formulation.

The present invention further provides processes for the manufacturing of said formulations and dosage forms.

Furthermore, the present invention provides methods of administration and treatment of HIV infected patients or suffering from AIDS.

### DESCRIPTION OF THE DRAWINGS

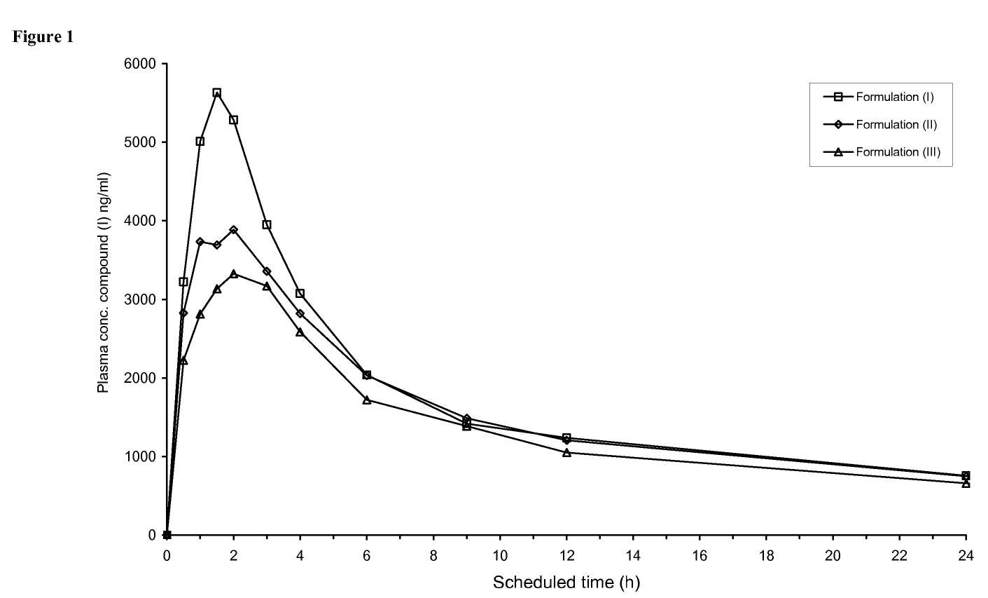

FIG. 1 shows the plasma concentration time curves of ethanolate form of compound (I) after a single intake under fasted conditions (boosted with ritonavir) in 3 self-microemulsifying drug delivery system formulations: formulation (I), formulation (II) and formulation (III) encapsulated in hard gelatin capsules.
Formulation (I): compound (I) ethanolate 108.40 mg, PVPK30 7.75 mg, Polyoxyl 40 Hydrogenated Castor oil 279.09 mg, Propylene glycol monocaprylate 186.06 mg, Purified diethylene glycol monoethyl ether 193.77 mg, Capsule size 00 (Licaps Swedish orange opaque).
Formulation (II): compound (I) ethanolate 162.6 mg, PVP K30 7.63 mg, Caprylocaproyl macrogol glyceride 473.9 mg, Lauryl macrogol glyceride 118.5 mg; Capsule size 00, (Licaps Swedish orange opaque).
Formulation (III): compound (I) ethanolate 162.6 mg, PVP K30 7.63 mg, Caprylocaproyl macrogol glyceride 533.13 mg, Lauryl macrogol glyceride 59.24 mg; Capsule size 00, (Licaps Swedish orange opaque).

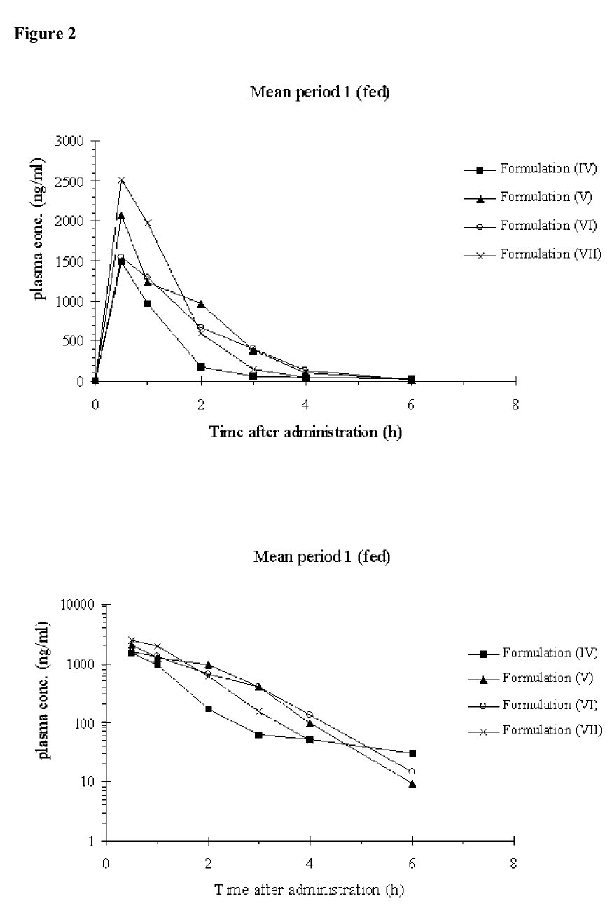

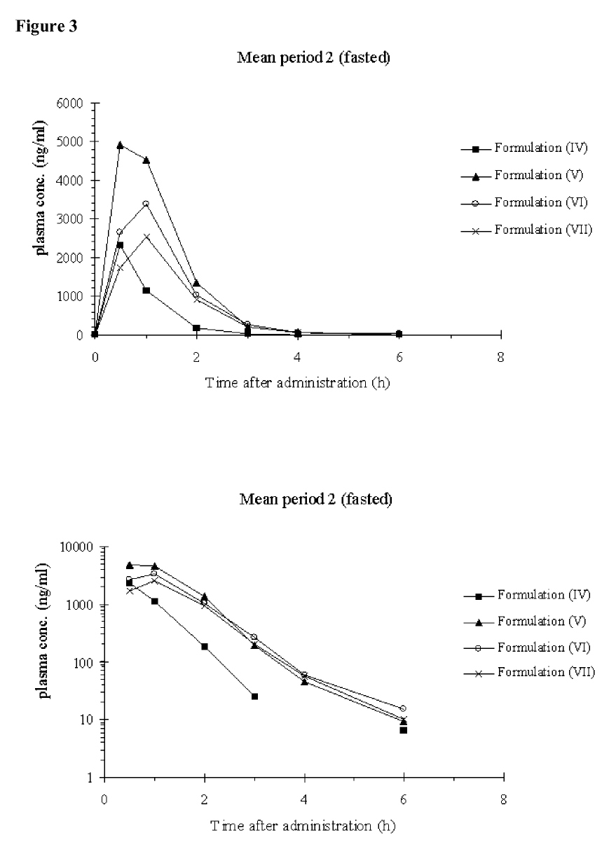

FIGS. 2 and 3 show the mean plasma concentrations of compound (I) in male dogs after single oral dosing of formulations at 100 mg/dog in period 1 (fed) and period 2 (fasted) respectively, for formulations (IV), (V), (VI), and (VII).
Formulation (IV): compound (I) ethanolate 108 mg, Caprylocaproyl macrogol-8 glycerides 372.75 mg, Lauryl macrogol-32 glycerides 62.1 mg, Purified diethylene glycol monoethyl ether 124.25 mg.
Formulation (V): D 6/4 of Example 4
Formulation (VI): E 8/2 of Example 4
Formulation (VII): E 9/1 of Example 4

### DETAILED DESCRIPTION OF THE INVENTION

The present invention provides a pharmaceutical formulation comprising a therapeutically effective amount of (3R,3aS,6aR)-hexahydrofuro[2,3-b]furan-3-yl (1S,2R)-3-[[(4-aminophenyl)sulfonyl](isobutyl)amino]-1-benzyl-2-hydroxypropyl-carbamate, salts, esters, polymorphic and pseudopolymorphic forms thereof; in association with a pharmaceutical carrier, said carrier comprising esters of alcohols with C₆₋₁₂ fatty acids or oils; a hydrophilic surfactant system; a nucleation inhibitor; and a hydrophilic solvent.

In particular, the present invention provides a pharmaceutical formulation comprising therapeutically effective amounts of compound (I), or pharmaceutically acceptable pseudopolymorphic forms thereof, in association with a pharmaceutical carrier, said carrier comprising a drug solubilizing effective amount of a propylene glycol ester of C₆₋₁₂ fatty acids; a hydrophilic surfactant system comprising at least one non-ionic surfactant, said non-ionic surfactant being present in an amount sufficient to form a microemulsion with the propylene glycol ester and drug when brought in contact with an aqueous medium; a nucleation inhibitor in a range of 0.1% (w/w) to 4% (w/w); and a hydrophilic solvent in a range of 1% (w/w) to 60% (w/w).

The pharmaceutical formulation of the present invention is a self-microemulsifying drug delivery system capable of forming an oil-in-water (o/w) microemulsion upon mixing with sufficient aqueous media. This microemulsion, once formed, comprises a mixture of a hydrophilic phase and a lipophilic phase. In the case of self-microemulsions or self-microemulsifying drug delivery systems, the aqueous media, i.e. hydrophilic phase, is provided by the human body, i.e. by the gastro-intestinal fluids in the GI tract. The microemulsion is made of substantially uniform and spherical droplets dispersed in a continuous medium. Microemulsions are characterized by their thermodynamic stability, optical clearness, i.e. substantially non-opaque, transparent or opalescent, and small average particle size in the submicron range, i.e. a diameter smaller than or equal to about 0.5 μm, preferably a diameter smaller than or equal to about 0.25 μm. The average particle size is dependant, amongst other factors, on the mixing speed with the aqueous media.

Self-microemulsifying drug delivery systems are also named as a self-microemulsifying preconcentrate, or as a self-microemulsifying formulation, all of which are considered equivalent terms in the present invention. Within the classification of pharmaceutical formulations, self-microemulsifying drug delivery systems are considered members of the family of self-emulsifying drug delivery systems, with the particularity of exhibiting a specific average particle size of the internal phase as mentioned hereinbefore. More information on self-emulsifying drug delivery systems or self-microemulsifying drug delivery systems can be found in C. W. Pouton, “Formulation of Self-Emulsifying Drug Delivery Systems”, Advanced Drug Delivery Reviews, 25 (1997) 47-58; which is incorporated herein by reference.

The term “carrier” is a term of art. As used herein, the term “carrier” refers to the composition that transports the drug across the biological membrane or within a biological fluid. In particular, the carrier of the present invention comprises the esters of alcohols with C₆₋₁₂ fatty acids or oils; the hydrophilic surfactant system comprising at least one non-ionic surfactant; the nucleation inhibitor; the hydrophilic solvent and optionally other adjuvants that normally are present therein, as described hereinbelow.

The drug formulated in the present invention is (3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate, and the pharmaceutically acceptable salts, esters, polymorphic and pseudopolymorphic forms thereof.

Psedopolymorphic forms of interest of compound of formula (I) are disclosed in WO 03/106461, incorporated herein by reference. In particular, pseudopolymorphic forms include the ethanolate, hydrate, methanolate, acetonate, dichloromethanate, ethylacetate solvate, 1-ethoxy-2-propanolate, anisolate, tetrahydrofuranate, isopropanolate, mesylate; in a ratio of compound to solvent ranging between (5:1) and (1:5), preferably in a ratio of compound to solvent of about 1:1. In a preferred embodiment, the drug is the ethanolate form of compound (I), or alternatively, the monohydrate and dihydrate forms thereof.

(3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate ethanolate is defined in terms of solubility as very slightly soluble according to Eur. Ph., and may be also defined as a lipophilic compound, or hydrophobic compound.

The term “lipophilic compound” refers to compounds with a log P around 2, a low intrinsic aqueous solubility (0.09-0.18 mg/ml) in the pH range of 2 to 6, and having a solubility in the self-microemulsifying formulation carrier of the present invention greater than or equal to 1 mg/ml. The log P value is measured by the compound's distribution behavior in a biphasic system such as the partition coefficient between the octanol and water phases; which is either determined experimentally or calculated by commercially available software.

The drug may be present in the self-microemulsifying drug delivery system formulation in a concentration around 2 to 80% (w/w) based on the total amount of the formulation. Preferably, the drug will be present in a concentration of 5 to 50%, more preferably from 10 to 30%, more preferably around 10, 12, 14, 16, 18, 20, 22, 25, 27, 28 or 30%.

The lipophilic phase component of the present self-microemulsifying drug delivery system formulation comprises esters of alcohols with C₆₋₁₂ fatty acids or oils; for example, such alcohols include ethylene glycol, propylene glycol, glycerol, polyethylene glycol, polypropylene glycol, sorbitol, pentaerythritol, and combinations and mixtures thereof.

Suitably, this lipophilic phase component encompasses polyethylene glycol fatty acid mono-, di-esters, and mixtures thereof, alcohol-oil transesterification products; polyethylene glycol glycerol fatty acid esters; mono- and diglycerides; polyglycerized fatty acids or polyglycerol esters of fatty acids; propylene glycol fatty acid esters; lower alcohol fatty acid esters.

Esters of glycerol with fatty acids may be monoglycerides, diglycerides and triglycerides. Esters with glycol-type alcohols will be monoesters and diesters. Both types of esters, mixtures and combinations thereof are meant in the definition of the lipophilic phase in the present invention. The terms “glycerol”, “glycerine” or “glycerin” are to be considered equivalent.

By C₆₋₁₂ fatty acids, it is meant saturated or unsaturated, linear or branch chained, substituted or unsubstituted fatty acids or fatty acid mixtures having from 6 to 12 carbon atoms and preferably those having eight 8 to ten 10 carbon atoms. Examples of C₆₋₁₂ fatty acids include for example caproic (6 carbon atoms), caprylic (8 carbon atoms), capric (10 carbon atoms), and lauric (12 carbon atoms) acids. Caprylic and capric acids are preferred.

A mixture of different C₆₋₁₂fatty acids may be used to be esterified to the alcohols, preferably two types of fatty acids are esterified to the alcohols, e.g. caprylic and capric acids, more preferably only one type of C₆₋₁₂fatty acid is esterified to the alcohols, e.g. caprylic acid.

The fatty acids chains may contain carbon-carbon double bonds. Preferably, the chain does not contain more than four carbon-carbon double bonds and more preferably no more than two carbon-carbon double bonds. Most preferably, the fatty acid chain contains no carbon-carbon double bonds. The fatty acids of the present invention may be branched, but it is preferred that a straight chain fatty acid is utilized. It is also preferred that the fatty acid contains an even number of carbon atoms.

A commonly used oil is castor oil or hydrogenated castor oil.

By the term “monoglyceride” is meant a fatty acid ester of glycerol having structural formula HO—CH₂—CH(OH)—CH₂—O—CO—R or HO—CH₂—CH(O—CO—R)—CH₂—OH, wherein R is an alkyl or alkenyl group having six to twelve carbon atoms. By the term “diglyceride” is meant a fatty acid ester of glycerol having structural formula HO—CH₂—CH(O—CO—R)—CH₂—O—CO—R or R—CO—O—CH₂—CH(OH)—CH₂—O—CO—R, wherein each R may be the same or different and is an alkyl or alkenyl group having six to twelve carbon atoms. By the term “triglyceride” is meant a fatty acid ester of glycerol having structural formula R—CO—O—CH₂—CH(O—CO—R)—CH₂—O—CO—R wherein each R may be the same or different and is an alkyl or alkenyl group having six to twelve carbon atoms. By the term “polyglycerized” is meant fatty acid esters of polyglycerol, which includes but is not limited to, diglycerols, triglycerols, tetraglycerols, and higher oligomeric glycerol polyethers.

The mono-, di-, and tri-glycerides may also be partially ethoxylated, wherein the free hydroxy groups are ethoxylated with ethylene glycol or ethylene oxide.

By polyethylene glycol (PEG) is meant a polymer having the general formula HO—(CH₂—CH₂—O)ₘ—H, where m represents the average number of oxyethylene groups. The number which follows PEG indicates the average molecular weight of the polymer. When m=1, an ethylene glycol or 1,2-dihydroxyethane is obtained.

By polypropylene glycol or PPG is meant a polymer having the general formula HO—(CH₂—CH₂—CH₂—O)ₙ—H, where n represents the average number of oxypropylene groups. The number which follows PPG indicates the average molecular weight of the polymer. When n=1, a propylene glycol or 1,3-dihydroxypropane is obtained, although the term propylene glycol refers as well to 1,2-dihydroxypropane, being the 1,2-dihydroxypropane the most preferred.

By the term “monoesters” is meant a fatty acid ester of PEG, PPG, ethylene glycol, or propylene glycol having structural formula R—CO—O—[(CH₂)₂₋₃—O]_{m/n}—H, or HO—[(CH₂)₂₋₃—O]_{m/n}—CO—R, wherein each R may be the same or different and is a monoalkyl, dialkyl, monoalkenyl, or dialkenyl group having six to twelve carbon atoms. By the term “diesters” is meant a fatty acid ester of PEG, PPG, ethylene glycol, or propylene glycol having structural formula R—CO—O—[(CH₂)₂₋₃—O]_{m/n}—CO—R, wherein each R may be the same or different and is a monoalkyl, dialkyl, monoalkenyl, dialkenyl group having six to twelve carbon atoms, or wherein the propylene glycol is 1,2-dihydroxypropane. The diester of the latter is R—CO—O—CH₂—CH(O—CO—R)—CH₃.

The lipophilic phase utilized in the invention is present in the self-microemulsifying drug delivery system in amounts sufficient to solubilize the lipophilic drugs in the pharmaceutical composition. Preferably the amounts present in the self-microemulsifying drug delivery system range from 2 to 90% (w/w) based on the total amount of self-microemulsifying drug delivery system, preferably in amounts between 2 and 70%, more preferably in amounts from 2 to 60%, even most preferably in amounts from 5 to 30%, such as around 8%, 12%, 16%, 20%, 22.4%, 23%, 24% or 27.5%.

The weight ratio of the drug to the lipohilic phase may range from about 1:0.5 to about 1:10, respectively, preferably ranges from about 1:1 to about 1:5, more preferably from about 1:1.5 to about 1:4, and most preferably, the drug and the lipophilic phase are present in a weight ratio of about 1:1.5 to 1:3.5.

Fatty acid esters of propylene glycol may be preferably used as a lipophilic phase in the present invention. In this class, propylene glycol monocaprylate (Capryol® 90, Gattefossé) is most preferred. It is a caprylic acid esterified product of propylene glycol containing at least about 90% monoester based on the total weight of propylene glycol ester, i.e., only one of the hydroxy groups is esterified. The term “ester of propylene glycol containing at least about 90% monoester by weight” means that at least 90% by weight up to a maximum of 100% of the esters formed in the esterification reaction is the monoester, although lower percentages of monoesters, such as 60%, 65%, 70%, 75%, 80% or 85% are also possible, and should not be limited in the scope of this invention.

Other preferred excipients suitable for use as lipophilic phases are Capmul® MCM, (Abitec Corp.), and Gelucire® 44/14 (Gattefossé).

Surfactants are surface-active amphiphilic compounds which facilitate emulsification when the lipophilic phase enters in contact with the hydrophilic phase. The term amphiphilic means that the compound has hydrophobic and hydrophilic portions. The surfactants suitable for use with the self-microemulsifying excipient formulation of the present invention are preferably hydrophilic. They may be ionic and non-ionic in nature, although non-ionic surfactants are preferred. By hydrophilic nature, it is meant surfactants capable of forming an oil-in-water (micro)emulsion.

The term “surfactant system” means a system comprising one or more surfactants. In practise, the surfactant system utilized in the present invention should possess an overall HLB value between 8 and 18 based on the HLB system. Preferably the HLB range for the surfactant system is between approximately 8 and 15, more preferably between approximately 9 to 11, even more preferably around 10, 10.1, 10.2, 10.3 or 10.4. An HLB value greater than 10 has been conventionally considered by the art as the cut-off vale for defining hydrophilic surfactants. Other reports consider an HLB range of 8-18 suitable for forming o/w microemulsions. Surfactants with any HLB value and still capable of forming o/w microemulsions are also suitable for the self-microemulsifying drug delivery system of the present invention. The surfactant system may therefore include one or more surfactants having a HLB lower than 10, or lower than 8, or more lipophilic in nature, as long as the final surfactant system is capable of forming an o/w emulsion, in particular an o/w microemulsion; or the overall HLB of the surfactant system is at least greater than 8. To calculate the final HLB value of the surfactant system, the method by Griffin (1949, 1954) may be used. Said method further allows the calculation of the relative quantities of the surfactants necessary to produce physically stable formulations for particular oil/water combinations.

Suitable surfactants for the present invention include but are not limited to polyethylene glycol fatty acid esters; alcohol-oil transesterification products; polyethylene glycol glycerol fatty acid esters; polyethylene glycol sorbitan fatty acid esters; polyethylene glycol alkyl ethers; polyethylene glycol alkyl phenols; poloxamers; mono- and diglycerides, polyglycerized fatty acids; sorbitan fatty acid esters, propylene glycol fatty acid esters; lower alcohol fatty acid esters; sterol and sterol derivatives; sugar esters; and ionic surfactants.

1. Polyethylene Glycol Fatty Acid Mono-, Di-esters, and Mixtures Thereof

Examples of this type of surfactants include, without being limited to, the following: PEG 4-100 monolaurate (Crodet L series, Croda); PEG 4-100 monooleate (Crodet O series, Croda); PEG 4-100 monostearate (Crodet S series, Croda, Myrj Series, Atlas/ICI); PEG 400 distearate (Cithrol 4 DS series, Croda); PEG 100, 200, 300 monolaurate (Cithrol ML series, Croda); PEG 100, 200, 300 monooleate (Cithrol MO series, Croda, Algon OL 60, Mosselman NV); PEG 400 dioleate (Cithrol 4 DO series, Croda); PEG 400-1000 monostearate (Cithrol MS series, Croda); PEG-4 laurate (Mapeg® 200 ML, PPG, Kessco® PEG 200 ML, Stepan, LIPOPEG 2 L, Lipo Chem.); PEG-4 oleate (Mapeg® 200 MO, PPG, Kessco® PEG 200 MO, Stepan); PEG-4 stearate (Kessco® PEG 200 MS, Stepan, Hodag 20 S, Calgene, Nikkol MYS-4, Nikko); PEG-5 stearate (Nikkol TMGS-5, Nikko); PEG-5 oleate (Nikkol TMGO-5, Nikko); PEG-6 oleate (Algon OL 60, Auschem SpA, Kessco® PEG 300 MO, Stepan, Nikkol MYO-6, Nikko, Emulgante A6, Condea); PEG-7 oleate (Algon OL 70, Auschem SpA); PEG-6 laurate (Kessco® PEG300 ML, Stepan); PEG-7 laurate (Lauridac 7, Condea); PEG-6 stearate (Kessco® PEG300 MS, Stepan); PEG-8 laurate (Mapeg® 400 ML, PPG, LIPOPEG 4 DL, Lipo Chem.); PEG-8 oleate (Mapeg® 400 MO, PPG, Emulgante A8 Condea); PEG-8 stearate (Mapeg® 400 MS, PPG, Myrj 45); PEG-9 oleate (Emulgante A9, Condea); PEG-9 stearate (Cremophor S9, BASF); PEG-10 laurate (Nikkol MYL-10, Nikko, Lauridac 10, Croda); PEG-10 oleate (Nikkol MYO-10, Nikko); PEG-10 stearate (Nikkol MYS-10, Nikko, Coster K100, Condea); PEG-12 laurate (Kessco® PEG 600 ML, Stepan); PEG-12 oleate (Kessco® PEG 600 MO, Stepan); PEG-12 ricinoleate; PEG-12 stearate (Mapeg® 600 MS, PPG, Kessco® PEG 600 MS, Stepan); PEG-15 stearate (Nikkol TMGS-15, Nikko, Koster K15, Condea); PEG-15 oleate (Nikkol TMGO-15, Nikko); PEG-20 laurate (Kessco® PEG 1000 ML, Stepan); PEG-20 oleate (Kessco® PEG 1000 MO, Stepan); PEG-20 stearate (Mapeg® 1000 MS, PPG, Kessco® PEG 1000 MS, Stepan, Myrj 49); PEG-25 stearate (Nikkol MYS-25, Nikko); PEG-32 laurate (Kessco® PEG 1540 ML, Stepan); PEG-32 oleate (Kessco® PEG 1540 MO, Stepan); PEG-32 stearate (Kessco® PEG 1540 MS, Stepan); PEG-30 stearate (Myrj 51); PEG-40 laurate (Crodet L40, Croda); PEG-40 oleate (Crodet 040, Croda); PEG-40 stearate (Myrj 52, Emerest® 2715, Henkel, Nikkol MYS-40, Nikko); PEG-45 stearate (Nikkol MYS-45, Nikko); PEG-50 stearate (Myrj 53); PEG-55 stearate (Nikkol MYS-55, Nikko); PEG-100 oleate (Crodet O -100, Croda); PEG-100 stearate (Myrj 59, Arlacel 165, ICI); PEG-200 oleate (Albunol 200 MO, Taiwan Surf.); PEG-400 oleate (LACTOMUL, Henkel, Albunol 400 MO, Taiwan Surf.); PEG-600 oleate (Albunol 600 MO, Taiwan Surf); PEG-4 dilaurate (Mapeg® 200 DL, PPG, Kessco® PEG 200 DL, Stepan, LIPOPEG 2-DL, Lipo Chem.); PEG-4 dioleate (Mapeg® 200 DO, PPG); PEG-6 dilaurate (Kessco® PEG 300 DL, Stepan); PEG-6 dioleate (Kessco® PEG 300 DO, Stepan); PEG-6 distearate (Kessco® PEG 300 DS, Stepan); PEG-8 dilaurate (Mapeg® 400 DL, PPG, Kessco® PEG 400 DL, Stepan, LIPOPEG 4 DL, Lipo Chem.); PEG-8 dioleate (Mapeg® 400 DO, PPG, Kessco® PEG 400 DO, Stepan, LIPOPEG 4 O, Lipo Chem.); PEG-8 distearate (Mapeg® 400 DS, PPG, CDS 400, Nikkol); PEG-10 dipalmitate (Polyaldo 2PKFG); PEG-12 dilaurate (Kessco® PEG 600 DL, Stepan); PEG-12 distearate (Kessco® PEG 600 DS, Stepan); PEG-12 dioleate (Mapeg® 600 DO, PPG, Kessco® 600 DO, Stepan); PEG-20 dilaurate (Kessco® PEG 1000 DL, Stepan); PEG-20 dioleate (Kessco® PEG 1000 DO, Stepan); PEG-20 distearate (Kessco® PEG 1000 DS, Stepan); PEG-32 dilaurate (Kessco® PEG 1540 DL, Stepan); PEG-32 dioleate (Kessco® PEG 1540 DO, Stepan); PEG-32 distearate (Kessco® PEG 1540 DS, Stepan); PEG-400 dioleate (Cithrol 4 DO series, Croda); PEG-400 distearate (Cithrol 4 DS series, Croda); PEG 4-150 mono, dilaurate (Kessco® PEG 200-6000 mono, dilaurate, Stepan); PEG 4-150 mono, dioleate (Kessco® PEG 200-6000 mono, dioleate, Stepan); PEG 4-150 mono, distearate (Kessco® 200-6000 mono, distearate, Stepan).

2. Alcohol-Oil Transesterification Products;

Most common oils used in this class are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. A preferred surfactant in this class is Cremophor RH40. Other examples comprise PEG-5, 9, and 16 castor oil (ACCONON CA series, ABITEC); PEG-20 castor oil (Emalex C-20, Nihon Emulsion, Nikkol CO-20 TX, Nikko); PEG-23 castor oil (Emulgante EL23); PEG-30 castor oil (Emalex C-30, Nihon Emulsion, Alkamuls® EL 620, Rhône-Poulenc, Incrocas 30, Croda); PEG-35 castor oil (Cremophor EL and EL-P, BASF, Emulphor EL, Incrocas-35, Croda, Emulgin RO 35, Henkel); PEG-38 castor oil (Emulgante EL 65, Condea); PEG-40 castor oil (Emalex C-40, Nihon Emulsion, Alkamuls® EL 719, Rhône-Poulenc); PEG-50 castor oil (Emalex C-50, Nihon Emulsion); PEG-56 castor oil (Eumulgin® PRT 56, Pulcra SA); PEG-60 castor oil (Nikkol CO-60TX, Nikko); PEG-100 castor oil, Thornley); PEG-200 castor oil (Eumulgin® PRT 200, Pulcra SA); PEG-5 hydrogenated castor oil (Nikkol HCO-5, Nikko); PEG-7 hydrogenated castor oil (Simusol® 989, Seppic, Cremophor WO7, BASF); PEG-10 hydrogenated castor oil (Nikkol HCO-10, Nikko); PEG-20 hydrogenated castor oil (Nikkol HCO-20, Nikko); PEG-25 hydrogenated castor oil (Simulsol® 1292, Seppic, Cerex ELS 250, Auschem SpA); PEG-30 hydrogenated castor oil (Nikkol HCO-30, Nikko); PEG-35 hydrogenated castor oil (Nikkol HCO-35, Nikko); PEG-40 hydrogenated castor oil (Cremophor RH 40, BASF, Croduret, Croda, Emulgin HRE, Henkel, Nikkol HCO-40, Nikko); PEG-45 hydrogenated castor oil (Cerex ELS 450, Auschem Spa); PEG-50 hydrogenated castor oil (Emalex HC-50, Nihon Emulsion, Nikkol HCO-50, Nikko); PEG-60 hydrogenated castor oil (Nikkol HCO-60, Nikko; Cremophor RH 60, BASF); PEG-80 hydrogenated castor oil (Nikkol HCO-80, Nikko); PEG-100 hydrogenated castor oil (Nikkol HCO-100, Nikko); PEG-8 corn oil (Labrafil® WL 2609 BS, Gattefossé); PEG-20 corn glycerides (Crovol M40, Croda); PEG-20 almond glycerides (Crovol A40, Croda); PEG-25 trioleate (TAGAT® TO, Goldschmidt); PEG-40 palm kernel oil, Crovol PK-70); PEG-60 corn glycerides (Crovol M70, Croda); PEG-60 almond glycerides (Crovol A70, Croda); PEG-8 caprylic/capric glycerides (Labrasol, Gattefossé, Labrafac CM 10, Gattefossé); PEG-6 caprylic/capric glycerides, Softigen® 767, Huls, Glycerox 767, Croda); Lauroyl macrogol-32 glyceride (Gelucire® 44/14, Gattefossé); Stearoyl macrogol glyceride (Gelucire® 50/13, Gattefossé).

Also included as oils in this category of surfactants are oil-soluble vitamin substances. The oil-soluble vitamin substances include vitamins A, D, E, K, and isomers, analogues, and derivatives thereof. The derivatives include organic acid esters of these oil-soluble vitamin substances, such as the esters of vitamin E or vitamin A with succinic acid. Thus, derivatives of these vitamins, such as tocopheryl PEG-1000 succinate (Vitamin E TPGS, available from Eastman) and other tocopheryl PEG succinate derivatives with various molecular weights of the PEG moiety, such as PEG 100-8000, are also suitable surfactants.

3. Polyethylene Glycol Glycerol Fatty Acid Esters

They include, amongst others, PEG-20 glyceryl laurate (Tagat® L, Goldschmidt); PEG-30 glyceryl laurate (Tagat® L2, Goldschmidt); PEG-15 glyceryl laurate (Glycerox L series, Croda); PEG-40 glyceryl laurate (Glycerox L series, Croda); PEG-20 glyceryl stearate (Capmul ER LMG, ABITEC, Aldo®, MS-20 KFG, Lonza); PEG-20 glyceryl oleate (Tagat® O, Goldschmidt); PEG-30 glyceryl oleate (Tagat® O2, Goldschmidt).

4. Polyethylene Glycol Sorbitan Fatty Acid Esters

Examples falling in this category are PEG-10 sorbitan laurate (Liposorb L-10, Lipo Chem.); PEG-20 sorbitan monolaurate (Tween-20, Atlas/ICI, Crillet 1, Croda, DACOL MLS 20, Condea); PEG-4 sorbitan monolaurate (Tween-2 1, Atlas/ICI, Crillet 11, Croda); PEG-80 sorbitan monolaurate (Hodag PSML-80, Calgene; T-Maz 28); PEG-6 sorbitan monolaurate (Nikkol GL-1, Nikko); PEG-20 sorbitan monopalmitate (Tween-40, Atlas/ICI, Crillet 2, Croda); PEG-20 sorbitan monostearate (Tween-60, Atlas/ICI, Crillet 3, Croda); PEG-4 sorbitan monostearate (Tween-61, Atlas/ICI, Crillet 31, Croda); PEG-8 sorbitan monostearate (DACOL MSS, Condea); PEG-6 sorbitan monostearate (Nikkol TS106, Nikko); PEG-20 sorbitan tristearate (Tween-65, Atlas/ICI, Crillet 35, Croda); PEG-60 sorbitan tetrastearate (Nikkol GS-460, Nikko); PEG-5 sorbitan monooleate (Tween-81, Atlas/ICI, Crillet 41, Croda); PEG-6 sorbitan monooleate (Nikkol TO-106, Nikko); PEG-20 sorbitan monooleate (Tween-80, Atlas/ICI, Crillet 4, Croda); PEG-40 sorbitan oleate (Emalex ET 8040, Nihon Emulsion); PEG-20 sorbitan trioleate (Tween-85, Atlas/ICI, Crillet 45, Croda); PEG-6 sorbitan tetraoleate (Nikkol GO-4, Nikko); PEG-30 sorbitan tetraoleate (Nikkol GO-430, Nikko); PEG-40 sorbitan tetraoleate (Nikkol GO-440, Nikko); PEG-20 sorbitan monoisostearate (Tween-120, Atlas/ICI, Crillet 6, Croda); PEG sorbitol hexaoleate (Atlas G-1086, ICI).

5. Polyethylene Glycol Alkyl Ethers

Ethers of polyethylene glycol and alkyl alcohols are suitable surfactants for use in the present invention. Exponents of this category include, amongst other, PEG-3 oleyl ether, oleth-3 (Volpo 3, Croda); PEG-5 oleyl ether, oleth-5 (Volpo 5, Croda); PEG-10 oleyl ether, oleth-10 (Volpo 10, Croda, Brij 96/97, Atlas/ICI); PEG-20 oleyl ether, oleth-20 (Volpo 20, Croda, Brij 98/99, Atlas/ICI); PEG-4 lauryl ether, laureth-4 (Brij 30, Atlas/ICI); PEG-9 lauryl ether); PEG-23 lauryl ether, laureth-23, (Brij 35, Atlas/ICI); PEG-10 cetyl ether (Brij 56, ICI); PEG-20 cetyl ether (Brij 58, ICI); PEG-10 stearyl ether (Brij 76, ICI); PEG-20 stearyl ether (Brij 78, ICI); PEG-100 stearyl ether (Brij 700, ICI).

6. Polyethylene Glycol Alkyl Phenols

Examples are for instance PEG-10-100 nonyl phenol (Triton X series, Rohm & Haas, Igepal CA series, GAF, Antarox CA series, GAF); PEG-15-100 octyl phenol ether (Triton N-series, Rohm & Haas, Igepal CO series, GAF, Antarox CO series, GAF).

7. Polyoxyethylene (POE)-Polyoxypropylene (POP) Block Copolymers or Poloxamers

The POE-POP block copolymers are a special class of polymeric surfactants. The structure of these surfactants, with hydrophilic POE and lipophilic POP moieties in well-defined ratios and positions, provides a wide variety of surfactants suitable for use in the present invention. These surfactants are available under various trade names, including Synperonic PE series (ICI); Pluronic® series (BASF), Emkalyx, Lutrol (BASF), Supronic, Monolan, Pluracare, and Plurodac. The generic term for these polymers is “poloxamer” (CAS 9003-11-6). These polymers have the formula:
HO(C₂H₄O)ₐ(C₃H₆O)_{b}(C₂H₄O)ₐH

wherein “a” and “b” denote the number of polyoxyethylene and polyoxypropylene units, respectively.

The compounds are listed by generic name, with the corresponding “a” and “b” values, such for example, Poloxamer 105 (a=11, b=16); Poloxamer 108 (a=46, b=16); Poloxamer 123 (a=7, b=21); Poloxamer 124 (a=11, b=21); Poloxamer 181 (a=3, b=30); Poloxamer 184 (a=13, b=30); Poloxamer 185 (a=19, b=30); Poloxamer 188 (a=75, b=30); Poloxamer 215 (a=24, b=35); Poloxamer 217 (a=52, b=35); Poloxamer 231 (a=16, b=39); Poloxamer 234 (a=22, b=39); Poloxamer 235 (a=27, b=39); Poloxamer 237 (a=62, b=39); Poloxamer 238 (a=97, b=39); Poloxamer 282 (a=10, b=47); Poloxamer 284 (a=21, b=47); Poloxamer 288 (a=122, b=47); Poloxamer 333 (a=20, b=54); Poloxamer 334 (a=31, b=54); Poloxamer 338 (a=128, b=54); Poloxamer 401 (a=6, b=67); Poloxamer 402 (a=13, b=67); Poloxamer 403 (a=21, b=67); Poloxamer 407 (a=98, b=67).

Other block co-polymers are also suitable for the present invention. The block co-polymers can be made of various block components in different combination and sequences, such as BA diblock, ABA triblock, BAB triblock, and other more complex combinations and sequences involving three or more block components. The block components can be any poly(alkylene oxide), poly(lactic acid), poly(glycolic acid), poly(lactic-co-glycolic acid), poly(vinylpyrrolidone) and poly(ε-caprolactone). The molecular weights of suitable block co-polymers can range from a few thousand to a few million Daltons. These block co-polymers can be either hydrophilic or lipophilic depending on the distribution and ratios of different block components. Other co-polymers, not necessarily block co-polymers, are also suitable for the present invention. The co-polymers can be made of monomers or of any combinations thereof. The monomer component can be any alkylene oxide, lactic acid, glycolic acid, vinylpyrrolidone, or ε-caprolactone.

Other poloxamers include tetrafunctional polyoxyethylene polyoxypropylene block copolymer of ethylene diamine, known as Poloxamine 908 (Tetronic 908®); Poloxamine 1307 (Tetronic 1307®); Poloxamine 1107 polyoxyethylene polyoxybutylene block copolymer, known as Polyglycol BM45®.

8. Mono- and Diglycerides

Although these surfactants are generally lipophilic, they may be included in the surfactant system in combination with more hydrophilic surfactants. Examples of these surfactants are the following:

Monopalmitolein (C16:1, Larodan); Monoelaidin (C18:1, Larodan); Monocaproin (C6, Larodan); Monocaprylin (Larodan); Monocaprin (Larodan); Monolaurin (Larodan); Glyceryl ricinoleate (Softigen® 701, Huls, HODAG GMR-D, Calgene, ALDO® MR, Lonza); Glyceryl monolaurate (Aldo® MLD, Lonza, Hodag GML, Calgene); Glycerol monostearate (Capmul® GMS, ABITEC, Myvaplex, Eastman, Imwitor® 191, Huls, Cutina® GMS, Aldo® MS, Lonza, Nikkol MGS series, Nikko); Glyceryl mono-, dioleate (Capmul® GMO-K, ABITEC); Glyceryl palmitic/stearic (Cutina MD-A, Estagel-G18); Glyceryl acetate (Lamegin® EE, Grunau GmbH); Glyceryl citrate/lactate/oleate/linoleate (Imwitor® 375, Huls); Caprylic/capric glycerides (Imwitor® 742, Huls); Lactic acid esters of mono, diglycerides (Lamegin GLP, Henkel); Dicaproin (C6, Larodan); Dicaprin (C10, Larodan); Dioctanoin (C8, Larodan); Dimyristin (C14, Larodan); Dipalmitin (C16, Larodan); Distearin (Larodan); Glycerol esters of fatty acids (Gelucire® 37/06, Gattefossé); Dipalmitolein (C16:1, Larodan); 1,2 and 1,3-diolein (C18:1, Larodan); Dielaidin (C18:1, Larodan); Dilinolein (C18:2, Larodan).

9. Polyglycerized Fatty Acids or Polyglycerol Esters of Fatty Acids

Examples include Polyglyceryl-2 stearate (Nikkol DGMS, Nikko); Polyglyceryl-2 oleate (Nikkol DGMO, Nikko); Polyglyceryl-2 isostearate (Nikkol DGMIS, Nikko); Polyglyceryl-3 oleate (Caprol® 3GO, ABITEC, Drewpol 3-1-O, Stepan); Polyglyceryl-4 oleate (Nikkol Tetraglyn 1-O, Nikko); Polyglyceryl-4 stearate (Nikkol Tetraglyn 1-S, Nikko); Polyglyceryl-6 oleate (Drewpol 6-1-O, Stepan, Nikkol 9 Hexaglyn 1-O, Nikko); Polyglyceryl-10 laurate (Nikkol Decaglyn 1-L, Nikko); Polyglyceryl-10 oleate (Nikkol Decaglyn 1-O, Nikko); Polyglyceryl-10 stearate (Nikkol Decaglyn 1-S, Nikko); Polyglyceryl-6 ricinoleate (Nikkol Hexaglyn PR-15, Nikko); Polyglyceryl-10 linoleate (Nikkol Decaglyn 1-LN, Nikko); Polyglyceryl-6 pentaoleate (Nikkol Hexaglyn 5-O, Nikko); Polyglyceryl-3 dioleate (Cremophor GO32, BASF); Polyglyceryl-3 distearate (Cremophor GS32, BASF); Polyglyceryl-4 pentaoleate (Nikkol Tetraglyn 5-O, Nikko); Polyglyceryl-6 dioleate (Caprol® 6G20, ABITEC, Hodag PGO-62, Calgene, Plurol Oleique CC 497, Gattefossé); Polyglyceryl-2 dioleate (Nikkol DGDO, Nikko); Polyglyceryl-10 trioleate (Nikkol Decaglyn 3-O, Nikko); Polyglyceryl-10 tetraoleate (Caprol IR 10G40, ABITEC, Hodag PGO-62, CALGENE, Drewpol 10-4-O, Stepan); Polyglyceryl-10 decaisostearate (Nikkol Decaglyn 10-IS, Nikko); Polyglyceryl-10 mono, dioleate (Caprol® PGE 860, ABITEC); Polyglyceryl polyricinoleate (Polymuls, Henkel).

10. Sorbitan Fatty Acid Esters

Sorbitan esters of fatty acids are hydrophobic surfactants but may still be used in the present invention, in combination with a hydrophilic surfactant. Typical examples of these surfactants are Sorbitan monolaurate (Span-20, Atlas/ICI, Crill 1, Croda, Arlacel 20, ICI); Sorbitan monopalmitate (Span-40, Atlas/ICI, Crill 2, Croda, Nikkol SP-10, Nikko); Sorbitan monooleate (Span-80, Atlas/ICI, Crill 4, Croda, Crill 50, Croda).

11. Propylene Glycol Fatty Acid Esters

Esters of propylene glycol and fatty acids are lipophilic surfactants still useful in the present invention in combination with hydrophilic surfactants. It will be noticed that this class of surfactants are also considered typically as components of the lipophilic phase.

Examples of this class of surfactants are, without being limited to, propylene glycol monocaprylate (Capryol® 90, Gattefossé, Nikkol Sefsol 218, Nikko); propylene glycol monolaurate (Lauroglycol 90, Gattefossé, Lauroglycol FCC, Gattefossé); propylene glycol oleate (Lutrol OP2000, BASF); propylene glycol myristate (Mirpyl); propylene glycol hydroxystearate; propylene glycol ricinoleate (Propymuls, Henkel); propylene glycol isostearate; propylene glycol monooleate, (Myverol P-O6, Eastman); propylene glycol dicaprylate/dicaprate (Captex® 200, ABITEC, Miglyol® 840, Huls, Neobee® M-20, Stepan); propylene glycol dioctanoate (Captex® 800, ABITEC); propylene glycol caprylate/caprate (Labrafac PG, Gattefossé); propylene glycol dilaurate; propylene glycol distearate (Kessco® PGDS, Stepan); propylene glycol dicaprylate (Nikkol Sefsol 228, Nikko); propylene glycol dicaprate (Nikkol PDD, Nikko).

12. Lower Alcohol Fatty Acid Esters

Esters of lower alcohols having 2-4 carbon atoms with long chained fatty acids, such as C₈₋₁₈fatty acids, may be as well suitable surfactants for use in the present invention. Exponents of this class include ethyl oleate (Crodamol EO, Croda, Nikkol EOO, Nikko); isopropyl myristate (Crodamol IPM, Croda); isopropyl palmitate (Crodamol IPP, Croda); ethyl linoleate (Nikkol VF-E, Nikko); isopropyl linoleate (Nikkol VF-IP, Nikko).

13. Sterol and Sterol Derivatives

A preferred sterol in this class of sterols and sterol derivatives is cholesterol or the esters of cholesterol with an organic acid, such cholesteryl succinate. Preferred sterol derivatives are those which include polyethylene glycol. These derivatives could be esters and ethers depending upon the chemical bonds formed between the polyethylene glycol moiety and the sterol moiety.

Examples include cholesterol, sitosterol, lanosterol; PEG-24 cholesterol ether (Solulan C-24, Amerchol); PEG-30 cholestanol (Nikkol DHC, Nikko); Phytosterol (Generol series, Henkel), PEG-25 phytosterol (Nikkol BPSH-25, Nikko); PEG-5 soya sterol (Nikkol BPS-5, Nikko); PEG-10 soya sterol (Nikkol BPS-10, Nikko); PEG-20 soya sterol (Nikkol BPS-20, Nikko); PEG-30 soya sterol (Nikkol BPS-30, Nikko).

14. Sugar Esters

This class may include sugar esters such as sucrose distearate/monostearate (Sucro Ester 11, Gattefossé, Crodesta F-110, Croda); sucrose dipalmitate; sucrose monostearate (Crodesta F-160, Croda); sucrose monopalmitate (Sucro Ester 15, Gattefossé; sucrose monolaurate (Saccharose monolaurate 1695, Mitsubishi-Kasei).

15. Ionic Surfactants

Alternatively ionic surfactants may be employed in the present invention. As such cationic, anionic and zwitterionic surfactants may be suitable hydrophilic surfactants for use in the present invention. Typical ionic surfactants are lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono- and di-acetylated tartaric acid esters of mono- and di-glycerides, citric acid esters of mono- and di-glycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

The above lists are only intended to serve as exemplification of surfactants that may be used in accordance with the present invention, and should not in any way be considered as exhaustive or as limiting the invention.

A suitable surfactant is PEG-40 hydrogenated castor oil, also known as POE (40) hydrogenated castor oil; and Polyoxyl 40 hydrogenated castor oil. PEG-40 hydrogenated castor oil is a PEG derivate of hydrogenated castor oil with an average of 40-45 moles of ethylene oxide. PEG-40 hydrogenated castor oil may be used as well as a solubilizer, wetting agent, and emollient for pharmaceuticals. It is commercially available under the trademarks of Cerex ELS 400, Cremophor® RH 40, Emalex HC-40, Eumulgin® HRE 40, Sabopal ELH 40, Simulsol® 1293, and Tagat® CH 40.

Another suitable surfactant is Vitamin E TPGS (d-alpha tocopheryl polyethylene glycol 1000 succinate), which may be preferably blended with Cremophor RH 40. Vitamin E TPGS is a water-miscible form of a vitamin E derivative that enhances drug solubility, permeability, and hence bioavailabilty. It is a pharmaceutically acceptable excipient.

In one embodiment of the invention, the surfactant system comprises Cremophor® RH 40 and Vitamin E TPGS in a ratio ranging between 5:1 to 1:5, respectively, preferably in a ratio ranging between 3:1 to 1:3, more preferably in a ratio ranging between 2:1 to 1:2, even more preferably in a ratio of about 1:1.

The weight ratio of the drug to the surfactant system may range from about 1:0.5 to about 1:9, more preferably from about 1:1 to about 1:6, more preferably from about 1:2 to about 1:5 and even more preferably from about 1:2.5 to about 1:4.8, and most preferably around 1:4.3.

The surfactant system represents from about 3% to about 90% by weight of the total composition, preferably from about 30% to about 90%, more preferably from 50% to 80%, most preferably around 55%, 57%, 60%, 62%, 70%, 75% or 80%. Said surfactant is present in an amount sufficient to form a microemulsion with the lipophilic drug and propylene glycol monoester when brought in contact with an aqueous medium.

It is to be noted in the present invention that the lipophilic phase may play a co-surfactant role in the excipient formulation. As used herein, the term “co-surfactant” means a component that can act either as a surfactant or as an emulsifier/solubilizer. The term co-surfactant denotes a cooperative surfactant function of the lipophilic phase in assisting the surfactant system described above in the formation of a microemulsion. Said co-surfactant may have a HLB value of less than 10. As such, the lipophilic phase may constitute as well, one of the surfactant members of the surfactant system, and therefore, the term “surfactant system” as referred in this invention, may include the lipophilic phase.

Thus, the polyethylene glycol fatty acid mono-, di-esters, and mixtures thereof; alcohol-oil transesterification products; polyethylene glycol glycerol fatty acid esters; mono- and diglycerides; polyglycerized fatty acids or polyglycerol esters of fatty acids; propylene glycol fatty acid esters; lower alcohol fatty acid esters, constituting the lipophilic phase, may have in addition a co-surfactant function.

The preferred lipophilic phase component, Capryol® 90 (GattefosséSA), also referred to as propylene glycol monocaprylate, or propylene glycol caprylate, may be used as a co-surfactant due to its solubilizing and surfactant properties. It is further a bioavailability enhancer, absorption enhancer for pharmaceutical liquid and capsule formulations, especially for poorly-soluble drugs; is also considered as a stabilizer for microemulsions. It is an oily liquid; with faint odor, and with a HLB value of 5.

Notwithstanding the co-surfactant role of the lipophilic phase, alternative compositions are possible wherein the co-surfactant is not necessarily a component of the lipophilic phase. In addition, the invention is not limited to one co-surfactant only. More than one co-surfactants are also permitted.

The total amount of cosurfactant or cosurfactants present in the self-microemulsifying drug delivery system of this invention, no matter their full correspondence with the lipophilic phase, is preferably from about 1.9 to about 60% (w/w), more preferably from about 3 to about 40% (w/w), even more preferably from 5 to 30% (w/w).

In one embodiment of the present invention, the ratio of the amount of the hydrophilic surfactant system and of the co-surfactant ranges from 1/9 to 9/1, meaning, from 1 part by weight of surfactant per 9 parts by weight of co-surfactant to 9 parts by weight of surfactant per 1 part by weight of co-surfactant. The invention has proved specially advantageous when the ratios between the hydrophilic surfactant system and the co-surfactant range between 6/4 and 9/1. Preferable ratios between the hydrophilic surfactant system and of the co-surfactant are 6/4, 7/3, 8/2, and 9/1.

The self-microemulsifying formulation of the present invention additionally includes a hydrophilic solvent, typically alcohols which are liquids at room temperature.

Suitable hydrophilic solvents may be short-chain alcohols, selected from ethanol, benzyl alcohol; alkylene glycols such as propylene glycol, 2-(2-ethoxyethoxy)ethanol (Transcutol®, Gattefossé), polypropylene glycol, polyethylene glycols such as polyethylene glycol 200, polyethylene glycol 300, polyethylene glycol 400, polyethylene glycol 600, polyethylene glycol 900, polyethylene glycol 540, polyethylene glycol 1450, polyethylene glycol 6000, polyethylene glycol 8000 and the like; glycerol; triacetin; propylene carbonate, dimethylisosorbide, Glycofurol; polyoxypropylene block copolymers, and mixtures thereof. A preferred pharmaceutically acceptable alcohol is Transcutol®.

Hydrophilic solvents are present in the formulation in a weight ratio based on the total weight of the composition of 1% to 60%, preferably from 2,9% to 50%, more preferably from 10% to 40%, even more preferably from 20% to 30% of the total weight of the composition. Hydrophilic solvents are present in the formulation in a weight to weight ratio in relation to the drug of about 2:1 to about 1:5 (drug:solvent), more preferably from about 1:1 to about 1:3, most preferably from about 1:1. to about 1:2.

The formulation of the present invention further encompasses a nucleation inhibitor, also referred herein as crystallization inhibitor, or crystal growth inhibitor. Nucleation inhibitors have the property of slowing the rate of precipitation or crystallization of the drug after the drug is initially dissolved. They may adjust certain properties in the formulation such as viscosity, osmolarity, and dielectric constant; acting as well as solubilizing agents.

Nucleation inhibitors are typically pharmaceutically acceptable polymers, which are soluble in aqueous solution at physiologically relevant pHs (e.g. 1-8). Neutral or ionizable polymer that have an aqueous-solubility of at least 0.1 mg/ml over a portion of the pH range of 1-8 may be suitable.

Polymers suitable for the formulation of the present invention may be synthetic products such as acrylic acid polymers, vinyl derivates; inorganic and mineral products; modified natural polymers, such as cellulosic and starch derivates; natural polymers. Non-polymeric nucleation inhibitors may also be suitable.

While specific polymers are listed as being suitable for use in the formulation of the present invention, blends of such polymers may also be suitable.

Preferably the nucleation inhibitor is selected under synthetic polymers, like polyvinyllactams, in particular polyvinylpyrrolidone (PVP); copolymers of vinyllactams, like N-vinylpyrrolidone, N-vinylpiperidone and N-vinyl-μ-caprolactam, but especially N-vinylpyrrolidone, with (meth) acrylic acid and/or (meth) acrylic esters, such as long-chain (meth) acrylates, e.g. stearyl (meth) acrylate, dialkylamino alkyl (meth) acrylates, which may be quaternized, and maleic anhydride, vinyl esters, in particular vinyl acetate, vinylformamide, vinylsulfonic acid or quaternized vinylimidazole; copolymers of vinyl acetate and crotonic acid; partially hydrolized polyvinyl acetate; polyvinyl alcohol; (meth)acrylic resins such as poly(hydroxyalkyl(meth)acrylates), poly(meth)acrylates, acrylate copolymers, e.g. from alkyl acrylates with (meth)acrylic acid, and copolymers of dimethylaminoethyl acrylates and methacrylic ester (e.g. Eudragit types); polyalkylene glycols such as polypropylene glycols and polyethylene glycols, preferably with molecular weights between 200 and 80000 (e.g. polyethylene glycol 4000); polyalkylene oxides, such as polypropylene oxides and, in particular polyethylene oxides, preferably of high molecular weight, especially with weight average molecular weights between 10,000 and 100,000; copolymers of methyl methacrylate and acrylic acid; polyacrylamides, polyvinylformamide (where appropriate partially or completely hydrolyzed);

Inorganic and mineral products include clays such as hydrated colloidal aluminum silicate clay (Bentonite®); aluminum silicate dihydrate (kaolin); fumed silica (Aerosil®).

Modified natural polymers encompass modified starches and modified celluloses, such as cellulose esters and, preferably cellulose ethers, e. g. methyl cellulose and ethyl cellulose, hydroxyalkylcelluloses, in particular hydroxypropylcellulose, hydroxyalkyl-alkylcelluloses, in particular hydroxypropylmethylcellulose or hydroxypropyl ethylcellulose, cellulose phthalates, in particular cellulose acetate phthalate and hydroxypropylmethylcellulose phthalate, starch degradation products, in particular starch saccharification products, such as maltodextrin.

Natural or predominantly natural polymers include, amongst others, gelatin, tragacanth gums, polyhydroxyalkanoates, e.g. polyhydroxybutyric acid and polylactic acid, polyaminoacids, e.g. polylysine, polyasparagine, polydioxanes and polypeptides, and mannans, especially galactomannans.

Non-polymeric nucleation inhibitors are also suitable such as polyols, for example those described in WO98/22094 and EP 0 435 450, especially sugar alcohols such as maltitol, mannitol, sorbitol, cellobiitol, lactitol, xylitol, erythritol and isomalt (Palatinit).

In particular, a preferable polymer is selected from polyvinylpyrrolidones, vinylpyrrolidone/vinyl acetate copolymers, hydroxyalkylcelluloses, hydroxyalkyl alkylcelluloses, cellulose phthalates, polyalkylene glycols, (meth)acrylic resins.

Most preferably the polymer of the present invention is polyvinylpyrrolidone (Kollidon®) with an average molecular weight between 3000 to about 500000, for example the polyvinylpyrrolidone with a molecular weight average between 7000 to about 60000, which includes Kollidon® 15, Kollidon® 17 PF, Kollidon® 25, Kollidon® 30; vinylpyrrolidone/vinyl acetate copolymers, such as Kollidon® VA 64, Kollidon® SR.

Nucleation inhibitors are present in the formulation in a weight ratio based on the total weight of the composition of 0.1% to 4%, preferably from 0.5% to 2%, more preferably from 0.5% to 1.5%, even more preferably from 0.9% to 1.3% of the total weight of the composition. Nucleation inhibitors are present in the formulation in a weight to weight ratio in relation to the drug of about 1:0.01 to about 1:0.1 (drug:nucleation inhibitor), more preferably from about 1:0.02 to about 1:0.09, most preferably from about 1:0.02. to about 1:0.07.

In an embodiment the self-microemulsifying drug delivery system of the present invention encompasses compound (I) ethanolate 14%, PVP K30 1%, Polyoxyl 40 Hydrogenated Castor oil 36%, Propylene glycol monocaprylate 24%, Purified diethylene glycol monoethyl ether 25%. In another embodiment the self-microemulsifying drug delivery system of the present invention encompasses compound (I) ethanolate 21.3%, PVP K30 1%, Caprylocaproyl macrogol glyceride 62.1%, Lauryl macrogol glyceride 15.5%. In yet another embodiment, the self-microemulsifying drug delivery system comprises compound (I) ethanolate 21.3%, PVP K30 1%, Caprylocaproyl macrogol glyceride 69.9%g, Lauryl macrogol glyceride 7.8%.

Suitable unit dosage forms that can be used in the present invention are, for example, hard gelatin capsules, soft gelatin capsules, tablets, caplets, enteric coated tablets, enteric coated hard gelatin capsules, enteric coated soft gelatin capsules, dragees, oral liquids, syrups, sprays, and suppositories. Soft gelatin capsules, hard gelatin capsules, enteric coated soft gelatin capsules, minicapsules, and syrops are preferred unit dosage forms, being soft gelatin capsules mostly preferred unit dosage forms. Gelatin capsules size may be 5, 4, 3, 2, 1, 0, 00, 000, preferably 0 and 00. The hard gelatin capsules which may be used in the present invention may be of different colours and of different closures types, such as the typical, Snap-Fit®, Coni-Snap® or Coni-Fit®, Coni-Snap Supro®, Licaps®. Amongst the soft gelatin capsules, capsulines, pearls, and globules are also included. A preferred hard gelatin capsule is Licaps®.

In general, the self-microemulsifying drug delivery system compositions of the present invention can be prepared in different orders of compounding. For instance, the lipophilic phase, the nucleation inhibitor and the hydrophilic solvent may be mixed at a temperature between 15° and 75° C., preferably between 20° and 60° C., either at room temperature, or higher. The drug is added and stirred until dissolved, followed by admixture of the surfactant system. Otherwise, the lipophilic phase is admixed with the drug, the hydrophilic solvent is added, followed by admixing of the nucleation inhibitor and the surfactant system. In each case, the skilled artisan will select a preferred order of mixing and the appropriate working temperatures to facilitate the homogeneous mixture of the self-microemulsifying drug delivery system components.

For the preparation of soft-gelatin capsules, the appropriate volume of the resulting mixture needed to provide the desired dose of the HIV protease inhibiting drug is filled into the soft-gelatin capsules. Various methods can be used for manufacturing and filling the soft elastic gelatin capsules, for example, a seamless capsule method, a rotary method (developed by Scherer) or a method using a Liner machine or an Accogel machine and the like. Also various manufacturing machines can be used for manufacturing the capsules. Typically, the soft elastic gelatin capsule is prepared by preparing the gel mass, encapsulating the fill material (forming, filling and sealing the capsule) and softgel drying.

The composition and preparation of the soft elastic gelatin capsule itself is well known in the art. The composition of a soft elastic gelatin capsule typically comprises from about 30% to about 50% by weight of gelatin NF, from about 10% to about 40% by weight of a plasticizer or a blend of plasticizers and from about 25% to about 40% by weight of water. Plasticizers useful in the preparation of soft elastic gelatin capsules are glycerin, sorbitol or sorbitol derivatives (for example, sorbitol-special and the like), propylene glycol, hexanetriol propylene carbonate, hexane glycol, sorbitans, tetrahydrofuryl alcohol ether, diethylene glycol monoethyl ether, 1,3-trimethyl-2-imidazolidone, dimethylisosorbide, and the like; or combinations thereof. However, it should be understood that the plasticizer which can be used in the present invention is not restricted to those mentioned above.

The soft elastic gelatin capsule material can also comprise additives such as preservatives, opacifiers, pigments, dyes or flavors and the like.

For the manufacture of tablets, coated tablets, dragees and hard gelatine capsules the protease inhibitors can be processed with pharmaceutically inert, inorganic or organic excipients. Lactose, maize starch or derivatives thereof, talc, stearic acid or its salts etc. can be used, for example, as such excipients for tablets, dragees and hard gelatine capsules.

Additives normally utilized in the pharmaceutical arts can also be added to the pharmaceutical composition and especially the carrier. These additives may be preserving agents, antioxidants, buffers, pigments, coloring agents, sweetening agents, flavoring agents, coating agents, granulating agents, disintegrants, glidants, lubricants, conventional matrix materials, complexing agents, absorbents, fillers. They may be used for customary purposes and in typical amounts without adversely affecting the properties of the compositions. The dosage forms of the present invention may also contain other therapeutically valuable substances.

Storage of the self-microemulsifying drug delivery system may be performed at low temperatures, as well as at room temperatures. Preferably storage is effected at cool conditions.

Compositions of the present invention are preferably administered to mammals, such as dog, cat, horse, pig, mice, rat and especially humans. The pharmaceutical compositions of the present invention are preferably suited for oral administration.

Oral unit dosage forms in accordance with the present invention will preferably contain from 10 mg to 1400 mg of drug, and more preferably from 50 to 800 mg, e.g., 50, 75, 100, 108.4, 150, 200, 216.8, 250, 300, 325.2, 350, 400, 450, 500, 550, 600, 650, 700, 750, 800 mg of drug. The dosage of the drug and the number of times administered to the patient will vary depending on several factors, the age of the patient, the severity of the condition of the patient, past medical history, among other factors, and will be determined by the physician in his sound discretion without an undue amount of experimentation.

### EXAMPLES

### Example 1

Preparation of Self-Microemulsifying Drug Delivery System Hard Gelatin Capsules

Compound (I) ethanolate was sieved to remove large material. 279.69 mg of Polyoxyl 40 Hydrogenated Castor oil were placed in a suitable vessel and were heated to 55-60° C. with continuous stirring. 7.75 mg of polyvinylpyrrolidone K30 (PVP K30) and 193.77 mg Purified diethylene glycol monoethyl ether were added to the vessel and stirred until dissolved. 108.4 mg of the sieved Compound (I) ethanolate was added slowly to the liquid by careful sprinkling it into the liquid while vigorously stirring and maintaining the temperature of the liquid at 55-60° C. 186.06 mg of Propylene glycol monocaprylate were then admixed to the previous liquid.

When all of the Compound (I) ethanolate had dissolved, the vessel was removed from the heat source, the stirring was stopped, and the resulting liquid was allowed to reach room temperature (about 20° C.). The cooled liquid was then filled into Licaps® Swedish orange opaque capsules.

A heating temperature of at least 55° C. was selected to prevent lengthy dissolution time of Compound (I) ethanolate in the lipophilic phase.

### Example 2

Internal Phase Particle Measurement

Particle size of various formulations was measured by the MicrotracUPA150 (10 nm-3 (m). Two placebo formulations both containing 40% Cremophor RH40, 30% Caproyl 90, and 30% Transcutol®, and one containing 1% PVP K30 were emulsified at varying mixing speeds and particle size was measured.

The procedure was as follows: a vial of the formulation and a beaker of 125 mL filtered deionoized water were heated in a 40° C. cabinet. They were removed from the cabinet, and the beaker was placed on a magnetic stir plate set at 300 rpm and 37° C. 0.5 mL of self-microemulsifying drug delivery system formulation were added with a syringe directly to the water phase over 15 seconds, followed by stirring during 10 minutes. The microemulsion was then brought into a Microtrac cell for particle size measurement.

TABLE 1
Cremophor RH40/Caproyl 90/Transcutol ®

| RPM | D₅₀ μm | Peak Vol % | SD μm |
|---|---|---|---|
| 100 | 0.0143 | 100 | 0.0037 |
| 300 | 0.0141 | 100 | 0.0039 |
| 1000 | 0.0143 | 100 | 0.0039 |

TABLE 2
Cremophor RH40/Caproyl 90/Transcutol ®/PVPK30

| RPM | D₅₀ μm | Peak Vol % | SD μm |
|---|---|---|---|
| 300 | 0.0144 | 100 | 0.0057 |
| 1000 | 0.0144 | 100 | 0.0031 |

Results indicated that mixing speed had no effect on particle size and distribution of placebo formulations. Next, particle size of compound (I) ethanolate in Formulation (I) according to the invention was measured at varying mixing speeds.

TABLE 3
Formulation (I)

| RPM | D₅₀ μm | Peak Vol % | SD μm |
|---|---|---|---|
| 100 | 0.2096 | 62 | 0.6780 |
| | 1.402 | 38 | |
| 300 | 0.2221 | 100 | 0.1078 |
| 1000 | 0.1926 | 87 | 0.0909 |
| | 0.0935 | 13 | |

Although mixing speed had minimal effect on the majority of small particles formed, it had a large impact on the particle size distribution and formation of additional particle sizes.

### Example 3

Ternary Diagram of Compound (I) Ethanolate Self-Microemulsifying Drug Delivery System

Based on solubility data, the following excipients were selected which could be used for the development of compound (I) ethanolate using the self-microemulsifying drug delivery system technology: Cremophor RH 40, Labrasol as surfactant and Capmul MCM, Capryol 90 and Gelucire 44/14 as co-surfactant. Transcutol P could be used as possible solvent.

TABLE 4
Qualitative composition of the different
formulations for the ternary diagram

| Formulation | Surfactant | Co-surfactant | Solvent |
|---|---|---|---|
| A | Cremophor RH 40 | Capmul MCM | — |
| B | Cremophor RH 40 | Capmul MCM | Transcutol P |
| C | Cremophor RH 40 | Capryol 90 | — |
| D | Cremophor RH 40 | Capryol 90 | Transcutol P |
| E | Labrasol | Gelucire 44/14 | — |
| F | Labrasol | Gelucire 44/14 | Transcutol P |
| G | Labrasol | Capmul MCM | — |
| H | Labrasol | Capmul MCM | Transcutol P |
| I | Labrasol | Capryol 90 | — |
| J | Labrasol | Capryol 90 | Transcutol P |

For each formulation the surfactant and co-surfactant were used in different ratios as indicated in following table.

TABLE 5
Ratios surfactant/co-surfactant

| Surfactant | Co-surfactant |
|---|---|
| 1 | 9 |
| 2 | 8 |
| 3 | 7 |
| 4 | 6 |
| 5 | 5 |
| 6 | 4 |
| 7 | 3 |
| 8 | 2 |
| 9 | 1 |

The batch size for each formulation was 1 g. Transcutol P was used at a concentration of 25% of the total excipient amount. Compound (I) ethanolate eq. 50 mg compound (I) was additionally added to the formulation (containing a surfactant, a co-surfactant and eventually a solvent—1 g).

The manufacturing directions for these formulations were as follows:
1. Melt the solid phase (Cremophor RH 40 or Gelucire 44/14) at 60° C.
2. Heat the liquid phase (Capryol 90, Labrasol or Capmul MCM) at 60° C. and mix it eventually with Transcutol P at 60° C.
3. Mix (1) and (2) to homogeneous at 60° C.
4. Dissolve compound (I) ethanolate in the solution (3), keeping the temperature at 60° C. and mix until a clear solution is obtained.
5. Keep the solution (4) at 37° C.

These formulations were used to set up a ternary diagram to evaluate which formulations stayed clear by adding demineralised water at a temperature of 37° C. to the compound (I) ethanolate self-microemulsifying drug delivery system formulations, by stirring at 37° C. In table 3, formulations which stayed clear are shown.

TABLE 6
Clear ternary diagram compound (I) ethanolate formulations and results
of the corresponding particle size distribution measurements
(μm) (Mastersizer S long bed)

| Formulation | Ratio S/CoS | D (v, 0.1) | D (v, 0.5) | D (v, 0.9) |
|---|---|---|---|---|
| A | 6/4 | 73.95 | 254.19 | 407.49 |
| A | 7/3 | 0.55 | 338.88 | 635.77 |
| A | 8/2 | 279.44 | 497.48 | 770.68 |
| A | 9/1 | 3.15 | 29.96 | 176.01 |
| B | 6/4 | 0.19 | 0.51 | 369.53 |
| B | 7/3 | 64.74 | 315.24 | 643.77 |
| B | 8/2 | 141.55 | 295.18 | 539.65 |
| B | 9/1 | 25.51 | 180.54 | 547.68 |
| C | 6/4 | 0.13 | 0.49 | 25.44 |
| C | 8/2 | 0.23 | 2.98 | 118.49 |
| C | 9/1 | 48.47 | 328.65 | 688.54 |
| D | 6/4 | 0.09 | 0.25 | 0.88 |
| D | 7/3 | 0.16 | 0.42 | 465.70 |
| D | 8/2 | 0.35 | 258.17 | 663.35 |
| D | 9/1 | 60.75 | 311.20 | 635.26 |
| E | 1/9 | 3.69 | 222.54 | 367.69 |
| E | 2/8 | 128.29 | 234.84 | 350.81 |
| E | 3/7 | 184.31 | 297.57 | 450.40 |
| E | 4/6 | 335.38 | 374.44 | 431.59 |
| E | 5/5 | 2.92 | 542.55 | 699.57 |
| E | 6/4 | 0.10 | 0.26 | 0.82 |
| E | 7/3 | 0.09 | 0.21 | 0.47 |
| E | 8/2 | 0.09 | 0.20 | 0.47 |
| E | 9/1 | 0.09 | 0.20 | 0.43 |
| F | 1/9 | 142.88 | 266.05 | 403.82 |
| F | 2/8 | 146.31 | 273.83 | 438.79 |
| F | 3/7 | 145.67 | 310.04 | 557.46 |
| F | 4/6 | 0.80 | 106.79 | 339.69 |
| F | 5/5 | 0.13 | 0.49 | 189.03 |
| F | 6/4 | 0.10 | 0.24 | 0.59 |

Formulations with particles beneath or about 500 nm were selected to carry out particle size distribution measurements: D6/4, E6/4, E7/3, E8/2, E9/1 and F6/4. The result of the particle size distribution is volume based.

TABLE 7
Qualitative composition of the selected formulations

| | | | | Ratio |
|---|---|---|---|---|
| Formulation | Surfactant | Co-surfactant | Solvent | S/CoS |
| D | Cremophor | Capryol 90 | Transcutol P | 6/4 |
| | RH 40 | | | |
| E | Labrasol | Gelucire 44/14 | — | 6/4 |
| E | Labrasol | Gelucire 44/14 | — | 7/3 |
| E | Labrasol | Gelucire 44/14 | — | 8/2 |
| E | Labrasol | Gelucire 44/14 | — | 9/1 |
| F | Labrasol | Gelucire 44/14 | Transcutol P | 6/4 |

To measure the size of the submicron particles, the selected formulation were submitted to further measurement with the Malvern Autosizer 4700. Peak analysis was done by intensity, volume and number.

TABLE 8
Particle size distribution (nm) (Malvern Autosizer 4700)

| | | Peak | | |
|---|---|---|---|---|
| Formulation | Ratio S/CoS | analysis | Mean ± Std | Width ± Std |
| D | 6/4 | intensity | 20.74 ± 0.46 | 13.30 ± 5.17 |
| | | volume | 17.72 ± 2.82 | 11.98 ± 0.99 |
| | | number | 15.56 ± 4.36 | 8.56 ± 2.62 |
| E | 6/4 | intensity | 318.78 ± 10.94 | 257.98 ± 71.48 |
| | | volume | 423.44 ± 25.57 | 213.36 ± 18.06 |
| | | number | 108.00 ± 90.67 | 53.34 ± 53.78 |
| E | 7/3 | intensity | 230.90 ± 11.63 | 143.02 ± 86.19 |
| | | volume | 137.62 ± 59.45 | 96.92 ± 60.83 |
| | | number | 118.94 ± 44.60 | 61.80 ± 29.55 |
| E | 8/2 | intensity | 155.70 ± 5.96 | 86.68 ± 40.61 |
| | | volume | 142.18 ± 9.13 | 84.54 ± 15.54 |
| | | number | 92.26 ± 63.67 | 44.50 ± 36.69 |
| E | 9/1 | intensity | 177.78 ± 7.64 | 103.34 ± 53.41 |
| | | volume | 165.92 ± 8.24 | 101.62 ± 33.61 |
| | | number | 125.10 ± 49.16 | 60.42 ± 29.66 |
| F | 6/4 | intensity | 281.32 ± 20.29 | 105.02 ± 52.66 |
| | | volume | 307.50 ± 44.25 | 135.46 ± 76.04 |
| | | number | 188.54 ± 90.30 | 106.90 ± 66.76 |

### Example 4

Optimisation of Compound (I) Ethanolate Self-Microemulsifying Drug Delivery System Formulations (Using PVP K30)

Based on the results obtained by the ternary diagram and particle size distribution measurements of compound (I) ethanolate emulsions (see Example 4), formulations D6/4, E6/4, E7/3, E8/2, E9/1 and F6/4 were selected for optimisation with PVP K30 nucleation inhibitor.

The batch size for each formulation was 10 g. Transcutol P was used at a concentration of 25%. PVP K30 was used in different concentrations: 0%, 0.5%, 1% and 1.5%. Compound (I) ethanolate eq. 100 mg, 150 mg, 200 mg, 250 mg, 300mg, 350 mg, 400 mg and 450 mg compound (I) was additionally added to the formulation (containing a surfactant, a co-surfactant, eventually solvent and eventually PVP K30-10 g). The manufacturing directions for these formulations were as follows:
1. Melt the solid phase (Cremophor RH 40 or Gelucire 44/14) at 60° C.
2. Heat the liquid phase (Capryol 90 or Labrasol) at 60° C. and mix it eventually with Transcutol P at 60° C.
3. Mix (1) and (2) to homogeneous at 60° C.
4. Add PVP K30 to the above solution by stirring at 60° C., to obtain a clear solution. Mix additionally 10 minutes.
5. Dissolve TMC114ethanolate in the solution (4), keeping the temperature at 60° C. and mix until a clear solution is obtained.
6. Keep the solution (5) at 37° C.

Particle size distribution measurements were carried out on the formulations where PVP K30 and compound (I) ethanolate could be dissolved by stirring at 60° C. during 24 hours, immediately after manufacturing. The formulations were filled in Licaps size 00 (transparent) in order to evaluate possible crystallisation of compound (I) ethanolate. Microscopic evaluation was done by observing the contents of the capsules after 2 week storage at ambient conditions. The result of the particle size distribution is volume based.

TABLE 9
Particle size distribution (μm) (Mastersizer S long bed) of compound (I)
ethanolate self-microemulsifying drug delivery system formulations
with 1% PVP K30

| | Ratio | Compound (I) | | | D |
|---|---|---|---|---|---|
| Formulation | S/CoS | ethanolate | D (v, 0.1) | D (v, 0.5) | (v, 0.9) |
| D | 6/4 | eq. 100 mg | 0.13 | 0.34 | 482.08 |
| D | 6/4 | eq. 150 mg | 0.09 | 0.24 | 0.74 |
| D | 6/4 | eq. 200 mg | 0.11 | 0.32 | 16.67 |
| D | 6/4 | eq. 250 mg | 0.21 | 11.11 | 87.57 |
| D | 6/4 | eq. 300 mg | 0.14 | 0.70 | 87.81 |
| D | 6/4 | eq. 350 mg | 0.12 | 0.41 | 53.22 |
| E | 6/4 | eq. 100 mg | 0.09 | 0.22 | 0.55 |
| E | 6/4 | eq. 150 mg | 0.09 | 0.20 | 0.41 |
| E | 6/4 | eq. 200 mg | 0.08 | 0.19 | 0.40 |
| E | 6/4 | eq. 250 mg | 0.09 | 0.20 | 0.42 |
| E | 7/3 | eq. 100 mg | 0.09 | 0.21 | 0.49 |
| E | 7/3 | eq. 150 mg | 0.09 | 0.20 | 0.40 |
| E | 7/3 | eq. 200 mg | 0.08 | 0.19 | 0.40 |
| E | 7/3 | eq. 250 mg | 0.09 | 0.21 | 0.47 |
| E | 8/2 | eq. 100 mg | 0.09 | 0.22 | 0.58 |
| E | 8/2 | eq. 150 mg | 0.09 | 0.19 | 0.40 |
| E | 8/2 | eq. 200 mg | 0.09 | 0.19 | 0.41 |
| E | 8/2 | eq. 250 mg | 0.09 | 0.21 | 0.52 |
| E | 9/1 | eq. 100 mg | 0.09 | 0.20 | 0.43 |
| E | 9/1 | eq. 150 mg | 0.08 | 0.19 | 0.40 |
| E | 9/1 | eq. 200 mg | 0.09 | 0.20 | 0.43 |
| E | 9/1 | eq. 250 mg | 0.09 | 0.21 | 0.50 |
| E | 9/1 | eq. 300 mg | 0.09 | 0.22 | 0.59 |
| E | 9/1 | eq. 350 mg | 0.09 | 0.22 | 0.58 |
| E | 9/1 | eq. 400 mg | 0.10 | 0.26 | 2.97 |
| F | 6/4 | eq. 100 mg | 0.09 | 0.23 | 0.61 |
| F | 6/4 | eq. 150 mg | 0.09 | 0.20 | 0.45 |
| F | 6/4 | eq. 200 mg | 0.09 | 0.20 | 0.44 |
| F | 6/4 | eq. 250 mg | 0.09 | 0.22 | 0.54 |
| F | 6/4 | eq. 300 mg | 0.11 | 0.31 | 52.91 |
| F | 6/4 | eq. 350 mg | 0.09 | 0.25 | 1.47 |
| F | 6/4 | eq. 400 mg | 0.10 | 0.27 | 7.46 |
| F | 6/4 | eq. 450 mg | 0.10 | 0.29 | 31.54 |

In formulations where Transcutol P was included (D6/4 and F6/4), the particle size increased with higher concentrations of compound (I). The particle size distribution in formulations without Transcutol P was not influenced by the concentration of compound (I).

To observe the distribution of the little particles, the formulation with compound (I) ethanolate eq. 200 mg compound (I) were submitted to further measurement with the Malvern Autosizer 4700. Peak analysis was done by intensity, volume and number.

TABLE 10
Particle size distribution (nm) (Malvern Autosizer 4700)

| | Ratio | Peak | | |
|---|---|---|---|---|
| Formulation | S/CoS | analysis | Mean ± Std | Width ± Std |
| D | 6/4 | Intensity | 235.88 ± 52.57 | 148.78 ± 92.26 |
| | | Volume | 493.26 ± 565.35 | 241.86 ± 264.83 |
| | | Number | 122.80 ± 83.10 | 47.50 ± 49.13 |
| E | 8/2 | Intensity | 233.20 ± 20.97 | 78.26 ± 72.30 |
| | | Volume | 195.26 ± 50.41 | 74.96 ± 60.47 |
| | | Number | 184.82 ± 48.83 | 63.52 ± 47.61 |
| E | 9/1 | Intensity | 238.94 ± 36.67 | 186.42 ± 82.60 |
| | | Volume | 96.96 ± 48.71 | 69.08 ± 52.13 |
| | | Number | 78.92 ± 34.36 | 38.52 ± 22.73 |

In formulation D6/4 no crystallisation had been detected up to a concentration of eq. 300 mg compound (I). No precipitation had been detected in formulation E6/4 and E7/3 up to a concentration of eq. 100 mg compound (I). In formulation E8/2 and E9/1 the concentration of compound (I) could be increased to eq. 250 mg, without precipitation of compound (I). No precipitation had been observed in formulation F6/4 up to a concentration of eq. 250 mg compound (I).

In FIGS. 2 and 3 mean plasma concentrations of compound (I) in male dogs after single oral dosing of formulations at 100 mg/dog in period 1 (fed) and period 2 (fasted) respectively, are shown for the formulations:
Formulation (IV): compound (I) ethanolate 108 mg, Caprylocaproyl macrogol-8 glycerides 372.75 mg, Lauryl macrogol-32 glycerides 62.1 mg, Purified diethylene glycol monoethyl ether 124.25 mg.
Formulation (V): D 6/4
Formulation (VI): E 8/2
Formulation (VII): E 9/1

1. A pharmaceutical formulation comprising (3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate, or salt, ester, polymorphic and pseudopolymorphic form thereof, in association with a pharmaceutical carrier, said carrier comprising esters of alcohols with C₆₋₁₂ fatty acids or oils; a hydrophilic surfactant system; a hydrophilic solvent; and a nucleation inhibitor.

2. A pharmaceutical formulation comprising (3R,3aS,6aR)-hexahydrofuro [2,3-b]furan-3-yl(1S,2R)-3-[[(4-aminophenyl)sulfonyl](isobutyl)amino]-1-benzyl-2-hydroxypropylcarbamate, or salt, ester, polymorphic and pseudopolymorphic form thereof, in association with a pharmaceutical carrier, said carrier comprising esters of alcohols with C₆₋₁₂ fatty acids or oils; a hydrophilic surfactant system; a hydrophilic solvent; and a nucleation inhibitor;
characterised in that the hydrophilic solvent is in a range of 1% (w/w) to 60% (w/w), and the nucleation inhibitor is in a range of 0.1% (w/w) to 4% (w/w) of the total formulation.

3. The pharmaceutical formulation according to claim 1, wherein the esters of alcohols with C₆₋₁₂ fatty acids or oils act as a co-surfactant.

4. The pharmaceutical formulation according to claim 3, wherein the ratio between the hydrophilic surfactant system and the co-surfactant ranges between 6/4 and 9/1.

5. The pharmaceutical formulation according to claim 1; wherein the esters of alcohols with C₆₋₁₂ fatty acids or oils are selected from propylene glycol monocaprylate, lauryl macrogol-32 glycerides, and mono- and diglycerides of C₈₋₁₀ fatty acids.

6. The pharmaceutical formulation according to claim 1, wherein the hydrophilic surfactant system comprising a mixture of 2 surfactants in a ratio of 3:1 to 1:3.

7. The pharmaceutical formulation according to claim 1, wherein the surfactants of the hydrophilic surfactant system are selected from the group of polyethylene glycol fatty acid esters; alcohol-oil transesterification products; polyethylene glycol glycerol fatty acid esters; polyethylene glycol sorbitan fatty acid esters; polyethylene glycol alkyl ethers; polyethylene glycol alkyl phenols; poloxamers; mono- and diglycerides, polyglycerized fatty acids; sorbitan fatty acid esters, propylene glycol fatty acid esters; lower alcohol fatty acid esters; sterol and sterol derivatives; sugar esters; and ionic surfactants.

8. The pharmaceutical formulation according to claim 1, wherein the surfactants of the hydrophilic surfactant system are selected from PEG-40 hydrogenated castor oil, d-alpha tocopheryl polyethylene glycol 1000 succinate, PEG-8 caprylic/capric glycerides, and mixtures thereof.

9. The pharmaceutical formulation according to claim 1, wherein the hydrophilic solvent is a short-chain alcohol.

10. The pharmaceutical formulation according to claim 1, wherein the nucleation inhibitor is selected from the group of synthetic products; inorganic and mineral products; modified natural polymers; natural polymers; and non-polymeric substances.

11. The pharmaceutical formulation according to claim 1, wherein the nucleation inhibitor is selected from the polyvinyllactams having a molecular weight between 3,000 and 500,000.

12. The pharmaceutical formulation according to claim 1, which comprises the ethanolate form of (3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate; Capryol® 90; a mixture of Cremophor RH40 and Vitamin E TPGS; Transcutol®, and PVP K30.

13. The pharmaceutical formulation according to claim 1, wherein the (3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate, or salt, ester, polymorphic and pseudopolymorphic form thereof is in a range of 5% (w/w) to 50% (w/w); the esters of alcohols with C₆₋₁₂ fatty acids or oils is in a range of 2% (w/w) to 60% (w/w); the hydrophilic surfactant system is in a range of 30% (w/w) to 90% (w/w); the hydrophilic solvent is in a range of 2.9% (w/w) to 50% (w/w); and the nucleation inhibitor is in a range of 0.1% (w/w) to 4% (w/w).

14. The pharmaceutical formulation according to claim 1, wherein the amount of (3R,3aS,6aR)-hexahydrofuro[2,3-b]furan-3-yl(1S,2R)-3-[[(4-aminophenyl)sulfonyl](isobutyl)amino]-1-benzyl-2-hydroxypropylcarbamate or salt, ester, polymorphic and pseudopolymorphic form thereof, is from 50 to 800 mg per unit dose.

15. The pharmaceutical formulation according to claim 1, wherein the formulation is in a form suitable for oral administration.

16. The pharmaceutical formulation according to claim 15, wherein the form suitable for oral administration is selected from soft gelatin capsules, hard gelatin capsules, enteric coated soft gelatin capsules, minicapsules, and syrups.

17. A method for the treatment of HIV infected patients or suffering from AIDS, whereby a pharmaceutical formulation according to claim 1 is administered to a patient in the need of such treatment.
